(19) United States
(12) Patent Application Publication
(10) Pub. No.: US 2020/0030198 A1
(43) Pub. Date: Jan. 30, 2020

(54) ANTIOXIDANT DISPERSION

(71) Applicant: FUJIFILM Corporation, Tokyo (JP)

(72) Inventor: Hiroyuki SUGIYAMA, Kanagawa (JP)

(73) Assignee: FUJIFILM Corporation, Tokyo (JP)

(21) Appl. No.: 16/587,757

(22) Filed: Sep. 30, 2019

Related U.S. Application Data
(63) Continuation of application No. PCT/JP2018/014329, filed on Apr. 3, 2018.

(30) Foreign Application Priority Data
Apr. 11, 2017 (JP) 2017-078150
Aug. 23, 2017 (JP) 2017-160072

(51) Int. Cl. A61K 8/04; A61K 8/34; A61K 8/36; A61K 8/73; A61K 8/92; A61K 8/37; A61K 8/67; A61K 47/10; A61K 47/14; A61K 47/44; A61K 47/36; A61K 9/10; A61K 47/22; A23L 3/3544; A23L 3/349

(52) U.S. Cl. CPC A61K 8/04; A23V 2002/00; A61K 8/361; A61K 8/732; A61K 8/922; A61K 8/375; A61K 8/676; A61K 8/678; A61K 47/10; A61K 47/14; A61K 47/44; A61K 47/36; A61K 9/10; A61K 47/22; A23L 3/3544; A23L 3/349; A61K 8/345

(57) ABSTRACT

An antioxidant dispersion contains an oil component having a polyunsaturated fatty acid content of less than 50% by mass, a water-soluble antioxidant, at least one kind of compound selected from the group consisting of a polyhydric alcohol compound having 5 or less carbon atoms and reduced starch syrup, an emulsifier, and water. The emulsifier preferably contain a compound having an HLB value of 4.0 or less. In addition, in the antioxidant dispersion, a content of the emulsifier is preferably 0.5% by mass to 20% by mass with respect to a total mass of the antioxidant dispersion.

### CROSS-REFERENCE TO RELATED APPLICATIONS

This application is a Continuation of International Application No. PCT/JP2018/014329 filed on Apr. 3, 2018, which claims priority to Japanese Patent Application No. 2017-078150 filed on Apr. 11, 2017 and Japanese Patent Application No. 2017-160072 filed on Aug. 23, 2017. The entire contents of these applications are incorporated herein by reference.

### BACKGROUND OF THE INVENTION

### 1. Field of the Invention

The present disclosure relates to an antioxidant dispersion capable of being suitably used in food, medicine, or cosmetics.

### 2. Description of the Related Art

In recent years, food including oils and fats as components, for example, processed food fried in oil such as fried cakes or instant noodles are greatly expanded. In addition to the development of container packaging for preventing oxidation of oils, the research regarding the using methods or components of the antioxidant in order to prevent oxidation due to oxygen dissolved in oils has also proceeded.

Materials disclosed in JP1998-174861A (JP-H10-174861A), JP1999-188256A (JP-H11-188256A), and JP4173927B are known as microcapsules or compositions of the related art.

JP1998-174861A (JP-H10-174861A) discloses a polynuclear type microcapsule having a W/O/W film structure obtained by applying a temperature change to a W/O/W type emulsion, which is formed by dispersing and emulsifying a W/O type emulsion particle having an internal water phase formed of a hydrophilic core material containing an active component for medicine or food into an oil phase containing oil and one or two or more kinds of emulsifier selected from the group consisting of higher fatty acid ester, polyglycerin higher fatty acid ester, polyglycerin condensed ricinoleic acid ester, and sucrose fatty acid polyester, to have an eternal water phase containing a hydrophilic film formation material formed of a water-soluble macromolecular compound, which becomes in a gelled state due to a further temperature change, to solidify the hydrophilic film formation material of the external water phase, and forming a microcapsule film of the hydrophilic film formation material by covering the W/O type emulsion particle.

JP1999-188256A (JP-H11-188256A) discloses an oil composition formed of a water-soluble active material and/or a water dispersible active material, a water phase containing polyhydric alcohol, and an oil phase containing an oil component and an emulsifier having HLB of 10 or less, in which the water phase is dispersed in the oil phase in a fine particle state, a content of the polyhydric alcohol in the water phase is 40% to 99% by weight with respect to a total weight of the water phase, and a content of water in the water phase is 30% by weight or less.

JP4173927B discloses a vitamin composition in which water-soluble vitamin having an average particle diameter equal to or smaller than 1 μm is W/O-dispersed in polyglycerin condensed ricinoleate ester and neutral lipid having a melting point equal to or lower than 45° C.

### SUMMARY OF THE INVENTION

An oil-soluble antioxidant that is used in food, medicine, or cosmetics containing oils and fats as a component in the related art has problems that the cost is high, compared to that of a water-soluble antioxidant, and thus, a large amount thereof cannot be used. Thus, the water-soluble antioxidant is desired to be used. In addition, the antioxidant of the related art may be desired to be changed, depending on preference of flavors.

However, it was difficult to exhibit a particularly excellent effect in an oil component and to obtain a dispersion having excellent emulsion stability, with the water-soluble antioxidant used in food and cosmetics, by the method of the related art.

An object to be achieved by one embodiment of the invention is to provide an antioxidant dispersion having high antioxidant capacity and excellent emulsion stability.

Specific means for achieving the above-mentioned object include the following embodiments.

<1> An antioxidant dispersion comprising: an oil component having a polyunsaturated fatty acid content of less than 50% by mass; a water-soluble antioxidant; at least one kind of compound selected from the group consisting of a polyhydric alcohol compound having 5 or less carbon atoms and reduced starch syrup; an emulsifier; and water.

<2> The antioxidant dispersion according to <1>, in which the emulsifier contains a compound having an HLB value of 4.0 or less.

<3> The antioxidant dispersion according to <2>, in which the emulsifier is a mixture of two kinds of compound having an HLB value of 4.0 or less or a mixture of a compound having an HLB value of 4.0 or less and a compound having an HLB value of 11.0 or more.

<4> The antioxidant dispersion according to any one of <1> to <3>, in which a content of the emulsifier is 0.5% by mass to 20% by mass with respect to a total mass of the antioxidant dispersion.

<5> The antioxidant dispersion according to any one of <1> to <4>, in which a content of the emulsifier is 3% by mass to 20% by mass with respect to a total mass of the antioxidant dispersion.

<6> The antioxidant dispersion according to any one of <1> to <5>, in which the oil component is at least one kind of compound selected from the group consisting of palm oil, rice oil, rapeseed oil, olive oil, sesame oil, safflower oil, coconut oil, and medium chain fatty acid triglyceride.

<7> The antioxidant dispersion according to any one of <1> to <6>, in which the oil component is at least one kind of compound selected from the group consisting of palm oil, rice oil, rapeseed oil, and medium chain fatty acid triglyceride.

<8> The antioxidant dispersion according to any one of <1> to <7>, in which the polyhydric alcohol compound having 5 or less carbon atoms is at least one kind of compound selected from the group consisting of glycerin, erythritol, threitol, arabinitol, xylitol, and libitol.

<9> The antioxidant dispersion according to any one of <1> to <8>, in which the emulsifier is glycerin fatty acid ester.

<10> The antioxidant dispersion according to any one of <1> to <9>, in which a content of the water-soluble antioxidant is 30% by mass or less with respect to a total mass of the oil component.

<11> The antioxidant dispersion according to any one of <1> to <10>, further comprising: an oil-soluble antioxidant.

<12> The antioxidant dispersion according to <11>, in which the oil-soluble antioxidant contains ascorbyl palmitate or tocopherol.

According to one embodiment of the invention, it is possible to provide an antioxidant dispersion having excellent emulsion stability.

### DESCRIPTION OF THE PREFERRED EMBODIMENTS

Hereinafter, specific embodiments of the disclosure will be described in detail, but the disclosure is not limited to the following embodiments, and modifications can be suitably performed within the scope of the object of the disclosure.

A range of numerical values shown using “to” in the disclosure means a range including numerical values before and after “to” as a minimum value and a maximum value.

A term “step” in the disclosure does not only mean an individual step, but also include a case in which a step cannot be clearly differentiated from another step, in a case where the desired object of the step is achieved.

In the disclosure, in a case where a plurality of substances corresponding to each component are present in a composition, the amount of each component in the composition means a content of the plurality of substances present in the composition, unless otherwise noted.

In the disclosure, a “water phase” is used in contrast with an “oil phase”, regardless of the type of a solvent.

In the disclosure, “% by mass” and “% by weight” are identical, and a “part by mass” and “part by weight” are identical.

In the disclosure, a combination of two or more preferred aspects is a more preferred aspect.

[Antioxidant Dispersion]

An antioxidant dispersion according to the disclosure contains an oil component having a polyunsaturated fatty acid content of less than 50% by mass; a water-soluble antioxidant; at least one kind of compound selected from the group consisting of a polyhydric alcohol compound having 5 or less carbon atoms and reduced starch syrup; an emulsifier; and water.

The inventors have thought that the oil component having a polyunsaturated fatty acid content of less than 50% by mass, the water-soluble antioxidant, at least one kind of compound selected from the group consisting of a polyhydric alcohol compound having 5 or less carbon atoms and reduced starch syrup, the emulsifier, and water are used in combination and concertedly act in the antioxidant dispersion, and accordingly, the antioxidant dispersion according to the disclosure becomes an antioxidant dispersion having excellent emulsion stability. Therefore, the antioxidant dispersion can be suitably used as an antioxidant dispersion containing a water-soluble antioxidant, and particularly, can be more suitably used in food, medicine, or cosmetics containing oils and fats as a component.

In addition, the antioxidant dispersion according to the disclosure also has excellent oxidation stability of the oil component contained, by using the aspect described above.

For example, as the invention disclosed in JP1998-174861A (JP-H10-174861A), the emulsion stability is deteriorated or the oxidation stability of the oil component contained is deteriorated, in a case where a polyhydric alcohol compound having 6 or more carbon atoms (for example, sorbitol) is used.

In addition, as the invention disclosed in JP1999-188256A (JP-H11-188256A), the emulsion stability is deteriorated or the oxidation stability of the oil component contained is deteriorated, in a case where an oil component having a polyunsaturated fatty acid content of 50% by mass or more (for example, soybean oil) is used.

The antioxidant dispersion according to the disclosure preferably contains a water phase and an oil phase and is more preferably an emulsion dispersion containing a water phase and an oil phase. In addition, the oil phase is preferably a continuous phase of the antioxidant dispersion.

In the antioxidant dispersion according to the disclosure, it is preferable that a large amount of the water-soluble antioxidant is contained in the water phase, rather than in the oil phase.

(Oil Component Having Polyunsaturated Fatty Acid Content of Less Than 50% by Mass)

The antioxidant dispersion according to the disclosure contains the oil component having a polyunsaturated fatty acid content of less than 50% by mass.

In the antioxidant dispersion according to the disclosure, the oil component is a component having a solubility to water at 25° C. of less than 0.1% by mass (less than 1 g/L) and indicates oils and fats such as triglyceride.

The antioxidant dispersion according to the disclosure may contain an oil component having a polyunsaturated fatty acid content of 50% by mass or more, in a case where the polyunsaturated fatty acid content of entire oil component contained is less than 50% by mass.

In the antioxidant dispersion according to the disclosure, the content of the oil component having a polyunsaturated fatty acid content of 50% by mass or more is preferably 20% by mass or less with respect to a total mass of the oil component, from viewpoints of emulsion stability and oxidation stability of oil component contained.

The polyunsaturated fatty acid content with respect to the total constituent fatty acid in the oil component of the disclosure can be determined by a method disclosed in “standard methods for test and analysis of oils and fats” published by Japan Oil Chemists' Society.

As the oil component, at least one kind selected from the group consisting of medium chain fatty acid triglyceride and animal and vegetable fats and oils is preferably contained, from viewpoints of emulsion stability, oxidation stability of oil component contained, and Food Sanitation Act.

As an example of medium chain fatty acid triglyceride, medium chain fatty acid triglyceride in which the number of carbon atoms of fatty acid configuring triglyceride is 8 to 10. The fatty acid of the medium chain fatty acid triglyceride is saturated fatty acid, in many cases, and in this case, the polyunsaturated fatty acid content is 0% by mass.

As the fatty acid having 8 to 10 carbon atoms, caprylic acid and capric acid are preferably used.

Examples of the animal and vegetable fats and oils include vegetable fats and oil such as palm oil (9.2% by mass), rice oil (rice bran oil, 33.3% by mass), rapeseed oil (26.1% by mass), olive oil (7.2% by mass), sesame oil (41.2% by mass), safflower oil (13.6% by mass (safflower oil having a high oleic acid content and a small polyunsaturated fatty acid content is preferably used, or a mixture of safflower oil having a high oleic acid content and safflower oil having a low oleic acid content may be used)), sunflower oil (6.8% by mass (sunflower oil having a high oleic acid content)), or coconut oil (1.5% by mass), and animal fat and oil such as liver oil (24.5% by mass), fish oil (33.3% by mass), beef tallow (3.6% by mass), or lard (9.8% by mass). The values in the brackets show examples of the polyunsaturated fatty acid content.

Among these, the oil component is preferably at least one kind of compound selected from the group consisting of palm oil, rice oil, rapeseed oil, olive oil, sesame oil, safflower oil (safflower oil having an oleic acid content of 50% by mass or more is preferable), coconut oil, and medium chain fatty acid triglyceride, and more preferably at least one kind of compound selected from the group consisting of palm oil, rice oil, rapeseed oil, and medium chain fatty acid triglyceride, from viewpoints of emulsion stability and oxidation stability of oil component contained.

The oil component may be used alone or in combination of two or more kinds thereof.

A content of the oil component is preferably 20% by mass to 50% by mass and more preferably 25% by mass to 50% by mass with respect to a total mass of the antioxidant dispersion, from viewpoints of emulsion stability and oxidation stability of oil component contained.

The content of the oil component is preferably 1 to 20 times, more preferably 2 to 15 times, and even more preferably 2 to 15 times the total mass of the water-soluble antioxidant, from viewpoints of emulsion stability and oxidation stability of oil component contained.

(Water-Soluble Antioxidant)

The antioxidant dispersion according to the disclosure contains the water-soluble antioxidant.

In the disclosure, the water-soluble antioxidant is defined as a compound having antioxidant capacity and the dissolved amount to water at 25° C. of 0.3% by mass or more.

The water-soluble antioxidant is not particularly limited and a well-known water-soluble antioxidant can be used.

As the water-soluble antioxidant, at least one kind of compound selected from the group consisting of ascorbic acid, ascorbic acid derivative, erythorbic acids, sulfites, tea polyphenol, gallic acid, Myrica rubra Extract, Pterocarpus santalinus Extract, Soy Extract, Black Tea Extract, Tea Extract, rose fruit Extract, and salt thereof is preferable, at least one kind of compound selected from the group consisting of ascorbic acid, ascorbic acid derivative, and salt thereof is more preferable, at least one kind of compound selected from the group consisting of ascorbic acid and ascorbic acid derivative is even more preferable, and ascorbic acid is particularly preferable, from viewpoints of emulsion stability and oxidation stability of oil component contained.

Specific examples of ascorbic acid and salt thereof used in the disclosure include ascorbic acid, sodium ascorbate, potassium ascorbate, calcium ascorbate, and L-ascorbic acid 2-glucoside.

Examples of ascorbic acid derivative and salt thereof used in the disclosure include ascorbic acid phosphate ester and salt thereof, ascorbic acid sulfate ester and salt thereof, and palmitate ascorbyl phosphate ester and salt thereof.

Among these, examples of ascorbic acid, ascorbic acid derivative, and salt thereof preferably include ascorbic acid, sodium ascorbate, potassium ascorbate, calcium ascorbate, and L-ascorbic acid 2-glucoside, from viewpoints of emulsion stability and oxidation stability of oil component contained.

The water-soluble antioxidant may be used alone or in combination of two or more kinds thereof.

A content of the water-soluble antioxidant can be suitably set, in accordance with the kind of the water-soluble antioxidant.

The content of the water-soluble antioxidant is preferably 0.1% by mass to 15% by mass, more preferably 0.1% by mass to 10% by mass, and even more preferably 0.1% by mass to 8% by mass, with respect to a total mass of the antioxidant dispersion, from viewpoints of antioxidant capacity as the dispersion, emulsion stability, and oxidation stability of oil component contained.

The content of the water-soluble antioxidant is preferably 30% by mass or less, more preferably 5% by mass to 30% by mass, and even more preferably 10% by mass to 25% by mass, with respect to a total mass of the oil component contained, from viewpoints of antioxidant capacity as the dispersion, emulsion stability, and oxidation stability of oil component contained.

(At Least One Kind of Compound Selected from The Group Consisting of Polyhydric Alcohol Compound Having 5 or Less Carbon Atoms and Reduced Starch Syrup)

The antioxidant dispersion according to the disclosure contains at least one kind of compound selected from the group consisting of a polyhydric alcohol compound having 5 or less carbon atoms and reduced starch syrup (hereinafter, also referred to as a “specific alcohol compound”).

As the polyhydric alcohol compound having 5 or less carbon atoms, a compound having 5 or less carbon atoms and 2 or more hydroxy groups may be used, and a compound having 3 to 5 carbon atoms and two to 5 hydroxy groups is more preferable, and a compound having 3 to 5 carbon atoms and 3 to 5 hydroxy groups is even more preferable, from viewpoints of emulsion stability and oxidation stability of oil component contained.

Specific examples of the polyhydric alcohol compound having 5 or less carbon atoms include glycerin, erythritol, threitol, arabinitol, xylitol, ribitol, propylene glycol, 1,3-butylene glycol, 1,2-pentanediol, ethylene glycol, diethylene glycol, and pentaerythritol.

Among these, at least one kind of compound selected from the group consisting of glycerin, erythritol, threitol, arabinitol, xylitol, ribitol, and propylene glycol is preferable.

As the polyhydric alcohol compound having 5 or less carbon atoms, a sugar alcohol having 5 or less carbon atoms is preferable, a compound represented by HOCH₂—(CHOH)ₙ—CH₂OH (n represents an integer of 1 to 3) is more preferable, at least one kind of compound selected from the group consisting of glycerin, erythritol, threitol, arabinitol, xylitol, and ribitol is even more preferable, and glycerin is particularly preferable, from viewpoints of emulsion stability and oxidation stability of oil component contained.

Particularly, glycerin is preferably used in the antioxidant dispersion according to the disclosure, because it is easy to decrease an average particle diameter of emulsified particles containing a water-soluble antioxidant and stably hold the particles having a small particle diameter for a long period of time.

In the same manner as described above, the reduced starch syrup is also preferably used in the antioxidant dispersion according to the disclosure, because it is easy to decrease an average particle diameter of emulsified particles containing a water-soluble antioxidant and stably hold the particles having a small particle diameter for a long period of time.

The reduced starch syrup is one kind of sugar alcohol and is synthesized (that is, hydrogenation) by reducing starch syrup. A viscosity of the reduced starch syrup can be adjusted by changing the composition of starch syrup (that is, saccharide) which is a raw material, and the reduced starch syrup is divided into high-saccharified reduced starch syrup and low-saccharified reduced starch syrup.

Among these, the low-saccharified reduced starch syrup is more preferably used, because it is easy to decrease an average particle diameter of emulsified particles containing a water-soluble antioxidant and stably hold the particles having a small particle diameter for a long period of time.

As the reduced starch syrup, starch syrup in which oligotose (linear oligosaccharide) is reduced is particularly preferable, from viewpoints of maintaining a particle diameter of emulsified particles containing a water-soluble antioxidant and preventing precipitation in the antioxidant dispersion.

The source of oligotose is not particularly limited, and linear oligosaccharide containing maltotriose (G3) as a main component (50% by mass or more) is preferable, and linear oligosaccharide containing maltotriose as a main component and having a low content of glucose and high-molecular-weight dextrin (preferably 10% by mass or less, more preferably 5% by mass or less) is more preferable.

The reduced starch syrup used in the disclosure may be obtained by the method described above or may be a commercially available product.

Examples of the commercially available product of the reduced starch syrup include OLIGOTOSE (manufactured by Sanwa Starch Co., Ltd.), OLIGOTOSE (product name, manufactured by B Food Science Co., Ltd.), ESWEE-30 (product name, manufactured by B Food Science Co., Ltd.), ESWEE-100 (product name, manufactured by B Food Science Co., Ltd.), OLIGOTOSE H-70 (product name, manufactured by Mitsubishi-Chemical Foods Corporation), HALLODEX (product name, manufactured by Hayashibara Co., Ltd.), TETRUP-H (product name, manufactured by Hayashibara Co., Ltd.), PENTRUP (product name, manufactured by Hayashibara Co., Ltd.), COUPLING SUGAR (product name, manufactured by Hayashibara Co., Ltd.), and COUPLING SUGAR-S (product name, manufactured by Hayashibara Co., Ltd.).

The reduced starch syrup used in the disclosure may be any of a solid content (powder) or a liquid containing a solvent (for example, water), and is preferably a liquid, from a viewpoint of productivity.

The commercially available liquid reduced starch syrup contains 20% by mass to 40% by mass of water, in many cases, but the content of water may be beyond the range, as long as the desired effect is obtained.

The specific alcohol compound is preferably a polyhydric alcohol compound having 5 or less carbon atoms, more preferably a sugar alcohol compound having 5 or less carbon atoms, and particularly preferably glycerin, from viewpoints of emulsion stability and oxidation stability of oil component contained.

The specific alcohol compound may be used alone or in combination of two or more kinds thereof.

A content of the specific alcohol compound is preferably 5% by mass to 50% by mass, more preferably 20% by mass to 50% by mass, and even more preferably 25% by mass to 45% by mass, with respect to a total mass of the antioxidant dispersion, from viewpoints of emulsion stability and oxidation stability of oil component contained.

The content of the specific alcohol compound is preferably 1 part by mass to 10 parts by mass, more preferably 2 parts by mass to 10 parts by mass, and even more preferably 3 parts by mass to 10 parts by mass, with respect to a total mass of the water-soluble antioxidant, from viewpoints of emulsion stability and oxidation stability of oil component contained.

(Emulsifier)

The antioxidant dispersion according to the disclosure contains an emulsifier.

The emulsifier used in the disclosure is not particularly limited, and is preferably a compound selected from the group consisting of glycerin fatty acid ester (for example, polyglycerin condensed ricinoleic acid ester, polyglycerin fatty acid ester, or glycerin fatty acid ester), sorbitan fatty acid ester, sucrose fatty acid ester, lecithin, saponin, sterol, and enzyme-degraded lecithin, more preferably a compound selected from the group consisting of glycerin fatty acid ester, sorbitan fatty acid ester, and sucrose fatty acid ester, even more preferably glycerin fatty acid ester, and particularly preferably polyglycerin condensed ricinoleic acid ester, from viewpoints of emulsion stability and oxidation stability of oil component contained.

As described above, the glycerin fatty acid ester of the disclosure also contains polyglycerin condensed ricinoleic acid ester and polyglycerin fatty acid ester.

Lecithin is a generic term of a mixture containing various phosphatides such as phosphatidyl choline (PC). Enzyme-degraded lecithin (also referred to as lysolecithin) is a composition containing lysophosphatidyl choline in which one fatty acid of a phosphatidyl choline molecule is removed by enzyme such as phospholipase. The enzyme-degraded lecithin used in the disclosure contains so-called hydrogenated enzyme-degraded lecithin having improved oxidation stability by performing a hydrogenation treatment and changing bonded fatty acid into saturated fatty acid.

The emulsifier used in the disclosure preferably contains a compound having a Hydrophile-Lipophile Balance (HLB) value of 4.0 or less and more preferably contains a compound having an HLB value of 2.0 or less, from a viewpoint of emulsion stability.

The emulsifier used in the disclosure more preferably contains a compound having an HLB value of 0.0 to 4.0 and even more preferably contains a compound having an HLB value of 1.0 to 4.0, from a viewpoint of emulsion stability.

The emulsifier used in the disclosure is more preferably a compound having an HLB value of 4.0 or less and even more preferably a compound having an HLB value of 2.0 or less, from a viewpoint of emulsion stability.

The emulsifier used in the disclosure is even more preferably a compound having an HLB value of 0.0 to 4.0, from a viewpoint of emulsion stability.

The HLB value generally means a balance between hydrophilicity and hydrophobicity used in a field of a surfactant. The HLB value can be calculated by using a calculation equation normally used, for example, Kawakami's Equation. In the specification, the following Kawakami's Equation is used.

HLB value=7+11.7 log(Mw/Mo)

Mw is a molecular weight of a hydrophilic group and Mo is a molecular weight of a hydrophobic group.

The emulsifier may be used alone or in combination of two or more kinds thereof.

In a case of two or more kinds, a combination of two or more kinds of compound having an HLB value of 4.0 or less is preferable, and a combination of a compound having an HLB value of 4.0 or less and a compound having an HLB value of 11.0 or more is also preferable.

As the compound having an HLB value of 11 or more, a compound having an HLB value of 15.0 or more is preferably used, and a compound having an HLB value of 15.0 to 20.0 is more preferably used.

A content of the emulsifier is preferably 0.5% by mass to 20% by mass, more preferably 3% by mass to 20% by mass, even more preferably 6% by mass to 20% by mass, and particularly preferably 8% by mass to 15% by mass, with respect to a total mass of the antioxidant dispersion, from viewpoints of emulsion stability and oxidation stability of oil component contained.

(Water)

The antioxidant dispersion according to the disclosure contains water.

The water is not particularly limited, as long as it is water that can be used in the antioxidant dispersion, and any of purified water, distilled water, ion exchange water, pure water, or ultrapure water such as milli Q water can be used. The milli Q water is ultrapure water obtained by a Milli Q Water Purification System which is an ultrapure water purification system manufactured by Merck KGaA.

A content of water in the antioxidant dispersion according to the disclosure is preferably 1 to 10 times, more preferably 2 to 7 times, and even more preferably 2 to 5 times the total mass of the water-soluble antioxidant, from viewpoints of emulsion stability and oxidation stability of oil component contained.

(Oil-Soluble Antioxidant)

The antioxidant dispersion according to the disclosure preferably further contains an oil-soluble antioxidant, from viewpoints of emulsion stability and oxidation stability of oil component contained.

The “oil-soluble antioxidant” in the disclosure may be a compound having a solubility to water at 25° C. of less than 0.3% by mass and functioning as an antioxidant, among various antioxidants disclosed in “Antioxidant Theory and Practice” (by Kajimoto, San Shobo, 1984), “Antioxidant Handbook” (by Saruwatari, Nishino, Tabata, Taiseisha, 1976), and carotenoid which will be described later.

Examples of the oil-soluble antioxidant used in the disclosure include vitamin E such as oil-soluble ascorbic acid ester which or tocopherol and a derivative thereof, vitamin A such as retinoic acid, retinol, retinol acetate, retinol palmitate, retinyl acetate, retinyl palmitate, or tocopheryl retinoic acid and a derivative thereof, a derivative of oil-soluble vitamin C, kinetin, retinoin, sesamin, α-lipoic acid, coenzyme Q10, flavonoids, di-n-butylhydroxytoluene (BHT), butyl hydroxyanisole (BHA), tretinoin, polyphenol, superoxide dismutase) SOD), and phytic acid.

The derivative of vitamin indicates a compound for forming vitamin by decomposition caused by pH, heat, light, oxidation, or enzyme. Among these, as the derivative, an esterified body is preferably used.

Among these, the oil-soluble antioxidant is preferably a compound selected from the group consisting of oil-soluble ascorbic acid ester, vitamin E, a derivative of vitamin E, SOD, phytic acid, retinoic acid, retinol, and retinol acetate, more preferably a compound selected from the group consisting of oil-soluble ascorbic acid ester, vitamin E, a derivative of vitamin E, retinoic acid, retinol, and retinol acetate, and even more preferably a compound selected from the group consisting of oil-soluble ascorbic acid ester, vitamin E, and a derivative of vitamin E, from viewpoints of emulsion stability and oxidation stability of oil component contained.

The oil-soluble antioxidant particularly preferably contains ascorbyl palmitate or tocopherol, from viewpoints of emulsion stability and oxidation stability of oil component contained.

The oil-soluble antioxidant may be used alone or in combination of two or more kinds thereof.

A content of the oil-soluble antioxidant is preferably 0.1% by mass to 15% by mass, more preferably 0.1% by mass to 10% by mass, and even more preferably 0.1% by mass to 8% by mass, with respect to a total mass of the antioxidant dispersion, from viewpoints of emulsion stability and oxidation stability of oil component contained.

(Other Components)

The antioxidant dispersion according to the disclosure may contain one kind or two or more kinds of any of other components, for example, additives such as a bioactive component such as a nutrient composition, an active component, or a pharmacological component, a dye, liquid sugar such as starch syrup, if necessary, in addition to each component described above. The component described above is not particularly limited, as long as it can be used in food, medicine, or cosmetics.

(Method for Producing Antioxidant Dispersion)

A method for producing the antioxidant dispersion according to the disclosure is not particularly limited, and the antioxidant dispersion is preferably produced by a producing method including preparing a mixed liquid obtained by mixing an oil phase composition containing an oil component and an emulsifier, and a water phase composition containing a water-soluble antioxidant, at least one kind of compound selected from the group consisting of a polyhydric alcohol compound having 5 or less carbon atoms and reduced starch syrup, and water with each other, and emulsifying the prepared mixed liquid by a typical method.

The mixed liquid to be provided for emulsifying may be prepared by individually preparing the oil phase composition and the water phase composition, respectively and then combining the prepared oil phase composition and water phase composition with each other, or may be prepared by collectively mixing or subsequently mixing the components included in the oil phase composition and the water phase composition with each other.

The oil phase composition preferably contains other random components (for example, oil-soluble antioxidant), together with the oil component and the emulsifier.

The water phase composition contains a water-soluble antioxidant, at least one kind of compound selected from the group consisting of a polyhydric alcohol compound having 5 or less carbon atoms and reduced starch syrup, and water.

In one preferred aspect of the method for producing the antioxidant dispersion according to the disclosure, for example, a) a water-soluble antioxidant, at least one kind of compound selected from the group consisting of a polyhydric alcohol compound having 5 or less carbon atoms and reduced starch syrup, and water are mixed with each other and dissolved to obtain a water phase composition, b) an oil component and an emulsifier are mixed with each other and dissolved to obtain an oil phase composition, and c) the water phase composition and the oil phase composition are mixed with each other while stirring to obtain a mixed liquid, and emulsification dispersion is performed with respect to the obtained mixed liquid.

In a case of the emulsification dispersion, for example, the producing method of using two or more kinds of emulsification devices by a method of performing the emulsification by using a stirrer, an impeller stirring, a homomixer, or a typical emulsification device using a shear action such as a continuous flow type shear device, and then providing the emulsified material to a high-pressure homogenizer, is preferable. In a case of using the high-pressure homogenizer, particle diameters of emulsified particles can be set to be substantially homogeneous. In order to realize further homogeneous particle diameter, the emulsification dispersion may be performed several times.

For the high-pressure homogenizer, there are a chamber type high-pressure homogenizer including a chamber having a fixed flow path of a treatment liquid, and a homogeneous valve type high-pressure homogenizer including a homogeneous valve.

In the homogeneous valve type high-pressure homogenizer, a width of a flow path of a treatment liquid can be easily adjusted, and accordingly, a pressure and a flow rate during the operation can be randomly set, and the operating range is wide. Thus, the homogeneous valve type high-pressure homogenizer can be preferably used in the producing of the antioxidant dispersion according to the disclosure.

The chamber type high-pressure homogenizer can also be suitably used for uses requiring ultrahigh pressure, because a mechanism of increasing the pressure is easily operated, although a degree of freedom of the operation is low.

Examples of the chamber type high-pressure homogenizer include MICROFLUIDIZER (manufactured by Microfluidics), nanomizer (manufactured by Yoshida Kikai Co., Ltd.), and ultimizer (manufactured by Sugino Machine Limited).

Examples of the homogeneous valve type high-pressure homogenizer include a Gaulin type homogenizer (manufactured by APV), a Rannie type homogenizer (manufactured by Rannie), a high-pressure homogenizer (manufactured by Niro Soavi), a homogenizer (manufactured by SANWA Machinery Trading Co., LTD.), a high-pressure homogenizer (manufactured by IZUMI FOOD MACHINERY), and an ultrahigh-pressure homogenizer (manufactured by Ika. Co., Ltd.).

The pressure of the high-pressure homogenizer is preferably 50 MPa or more, more preferably 50 MPa to 250 MPa, and even more preferably 100 MPa to 250 MPa.

It is preferable that the obtained antioxidant dispersion is cooled through any cooler, preferably within 30 seconds and more preferably within 3 seconds, immediately after passing the chamber, from a viewpoint of maintaining the particle diameter of the emulsified particles.

—Average Particle Diameter of Antioxidant Dispersion—

An average particle diameter of the antioxidant dispersion (volume average particle diameter of water phase) is not particularly limited, and is preferably 5 nm to 200 nm, and, from viewpoints of excellent transparency and stability of the antioxidant dispersion, is more preferably 10 nm to 150 nm, and even more preferably 10 nm to 120 nm.

The measurement of the average particle diameter of the antioxidant dispersion (volume average particle diameter of water phase) is preferably performed by a dynamic light scattering method. Examples of a commercially available measurement device using the dynamic light scattering method include NANOTRAC UPA (product name, manufactured by Nikkiso Co., Ltd.), a dynamic light scattering type particle size distribution measurement device LB-550 (product name, manufactured by Horiba, Ltd.), Fiber-Optics Particle Analyzer FPAR-1000 (product name, manufactured by Otsuka Electronics Co., Ltd.), and the measurement device is not limited thereto, as long as it can perform the measurement of the particle diameter based on the principle of the dynamic light scattering method.

The average particle diameter of the antioxidant dispersion, for example, can be measured by putting a 100-fold distilled antioxidant dispersion in a sample cell in a device, using a value measured at 25° C., and the measurement is performed with a 50% accumulated particle diameter using Fiber-Optics Particle Analyzer FPAR-1000 (product name) manufactured by Otsuka Electronics Co., Ltd.

(Use of Antioxidant Dispersion)

The antioxidant dispersion according to the disclosure can be used for various uses of food (functional food, health food, or drink), medicine, quasi-drugs, and cosmetics. Particularly, the antioxidant dispersion can be suitably used as the antioxidant of oil and fat for food.

Examples of dosage forms of food, medicine, quasi-drugs, and cosmetics, to which the antioxidant dispersion according to the disclosure is applied, include liquid preparation and solid preparation (powdered drug or granule).

The antioxidant dispersion according to the disclosure can be applied to the liquid preparation and solid preparation, for example, a hard capsule, a soft capsule, or a tablet. These preparations can be produced by further adding a diluting agent, a disintegrator, a binder, a lubricant, a surfactant, a water-soluble polymer, a sweetener, a flavor improvement, or an acidifier, according to the dosage form by the typical method. The coating or sugar coating of the solid preparation obtained by applying the antioxidant dispersion according to the disclosure may be performed by a well-known method.

### EXAMPLES

The disclosure will be described in more detail with reference to the following examples, and the disclosure is not limited to the examples. The antioxidant dispersion produced in the examples can be suitably applied to food (functional food or health food), medicine, quasi-drugs, or cosmetics. “%” and “part” is based on the mass, unless otherwise noted.

### Example 1

Water, glycerin, and ascorbic acid were mixed and dissolved with the amounts shown in Table 1, and a water phase composition was obtained.

Glycerin fatty acid ester (polyglycerin condensed ricinoleic acid ester) and edible fat and oil (oil component) were mixed and dissolved with the amounts shown in Table 1, and an oil phase composition was obtained.

The water phase composition and the oil phase composition obtained were mixed while stirring using a magnetic stirrer, and a mixed liquid was obtained.

While continuing the stirring with a stirrer, the mixed liquid was heated to 40° C. for 30 minutes, a shearing force was applied at 3,000 rpm (rotation/min) for 3 minutes by using a TK homomixer (manufactured by Primix Corporation), and an emulsified composition (dispersion) was prepared.

The obtained emulsified composition was subjected to a treatment three times at a pressure of 200 MPa by Star Burst Mini (manufactured by Sugino Machine Limited), and accordingly, an antioxidant dispersion of Example 1 was obtained. The antioxidant dispersion obtained in Example 1 was a water-in-oil (W/O) emulsified dispersion.

TABLE 1

| | |
|---|---|
| | Used amount |
| Component | (% by mass) |

Water phase composition

| | |
|---|---|
| Ascorbic acid | 5 |
| Glycerin | 42 |
| Water | 18 |

Oil phase composition

| | |
|---|---|
| Glycerin fatty acid ester | 10 |
| Edible fat and oil | 25 |
| Content of water phase and oil phase (% by mass) | 100 |

The details of each compound used in Example 1 are shown as follows.

Edible fat and oil: medium chain fatty acid triglyceride (product name O.D.O, triglyceryl (caprylate/caprate), manufactured by The Nisshin Oillio Group, Ltd., polyunsaturated fatty acid content: 0% by mass)

Glycerin (product name: food additive glycerin, manufactured by Kao Corporation)

Glycerin fatty acid ester (polyglycerin condensed ricinoleic acid ester, product name: POEM PR300, manufactured by Riken Vitamin Co., Ltd.)

Ascorbic acid (vitamin C, product name: L-ascorbic acid, manufactured by DSM)

### Example 2

Water, glycerin, and ascorbic acid were mixed and dissolved with the amounts shown in Table 1, in the same manner as in Example 1, and a water phase composition was obtained.

Glycerin fatty acid ester (polyglycerin condensed ricinoleic acid ester) and edible fat and oil (oil component) were mixed and dissolved with the amounts shown in Table 1, in the same manner as in Example 1, and an oil phase composition was obtained.

The water phase composition and the oil phase composition obtained were mixed at the same ratio as in Example 1, while stirring using a magnetic stirrer, and a mixed liquid was obtained.

While continuing the stirring with a stirrer, the mixed liquid was heated to 40° C. for 30 minutes, a shearing force was applied at 600 watt for 3 minutes by using an ultrasonic homogenizer US-600AT (manufactured by NISSEI Corporation), and an emulsified composition (dispersion) was prepared.

The obtained emulsified composition was subjected to a treatment three times at a pressure of 200 MPa by Star Burst Mini (manufactured by Sugino Machine Limited), and accordingly, an antioxidant dispersion of Example 2 was obtained. The antioxidant dispersion obtained in Example 2 was a water-in-oil (W/O) emulsified dispersion.

The details of each compound used in Example 2 are shown as follows.

Edible fat and oil: palm oil (product name, The Nisshin OilliO Group, Ltd., polyunsaturated fatty acid content: 9.2% by mass)

Glycerin (product name: food additive glycerin, manufactured by NOF CORPORATION)

Glycerin fatty acid ester (polyglycerin condensed ricinoleic acid ester, product name: POEM PR300, manufactured by Riken Vitamin Co., Ltd.)

Ascorbic acid (vitamin C, product name: L-ascorbic acid, manufactured by Fuso Chemical Co., Ltd.)

### Examples 3 to 10 and Comparative Examples 1 to 4

Antioxidant dispersions of Examples 3 to 10 and Comparative Examples 1 to 4 were produced in the same manner as in Example 1, except that each composition, and a use ratio of the water-soluble antioxidant and the oil component shown in Table 2 were changed.

All of the antioxidant dispersions obtained in Examples 3 to 10 were water-in-oil (W/O) emulsified dispersion.

<Evaluation of Antioxidant Dispersion>

Each obtained antioxidant dispersion was evaluated by the following method. The evaluation results are collectively shown in Table 2.

(Standard Oil and Fat Analysis Test (CDM Test)

2,000 ppm of a sample was added to palm oil to get 50 ml with an oxidation stability test device manufactured by Metrohm AG by a Rancimat method, and the measurement was performed at 120° C. and 180° C. 2,000 ppm of the antioxidant dispersion was added to palm oil to get 50 ml, this was subjected to the CDM test, and the time when an inflection point was approached was measured. A test result obtained with only the palm oil without adding the antioxidant was set as a standard, and the evaluation was performed. In a case of “Fr”, the obtained material within 7 days after the production was subjected to the measurement, and in a case of “elapse of time”, the obtained material after three months from the production (stored at 10° C. to 15° C.) was subjected to the measurement. The evaluation standard is as follows. In the following evaluation standard, A or B is a practically acceptable range.

A: approximately 120% or more of standard time

B: approximately 110% to 119% of standard time

C: approximately 105% to 109% of standard time

D: approximately 95% to 104% of standard time

(Emulsion Stability)

The obtained oxidant dispersion was stored in a cold storage (10° C. to 15° C.) for 2 months and at room temperature (25° C.) for 10 days. A state where the standard is not beyond the standard (external appearance standard: oil-soluble, white-clouded or yellow-clouded liquid having a viscosity and having a unique smell) and the oil-water separation that cannot be visually confirmed was evaluated with a 5 ml transparent glass vial bottle. The specific evaluation standard is as follows. In the following evaluation standard, A or B is a practically acceptable range.

A: precipitation is not recognized in the emulsified composition.

B: slight suspended matter is recognized in the emulsified composition.

C: deposition and precipitation are recognized in the emulsified composition.

D: precipitation in the emulsified composition is significant and phase separation is recognized.

TABLE 2

| | | | Polyhydric | | | | |
|---|---|---|---|---|---|---|---|
| | | | alcohol | | | | |
| | | | compound | | | | |
| | | Mass ratio of | having 5 or less | | | | |
| | | water-soluble | carbon atoms or | | | CDM test | |
| | Oil component | antioxidant/oil | reduced starch | | Emulsion | | Elapse |
| | (edible fat and oil) | component | syrup | Emulsifier | stability | Fr | of time |
| Comparative Example 1 | Soybean oil | ⅓ | Glycerin | Glycerin fatty | D | D | D |
| | | | | acid ester | | | |
| Comparative Example 2 | Medium chain fatty acid triglyceride | ¼ | Sorbitol | Glycerin fatty | D | D | B |
| | | | | acid ester | | | |
| Comparative Example 3 | Palm oil | ⅕ | Not used | Glycerin fatty | C | B | D |
| | | | | acid ester | | | |
| Comparative Example 4 | Medium chain fatty acid triglyceride | ¼ | Not used | Glycerin fatty | D | D | D |
| | | | | acid ester | | | |
| Example 1 | Medium chain fatty acid triglyceride | ⅕ | Glycerin | Glycerin fatty | A | A | A |
| | | | | acid ester | | | |
| Example 2 | Palm oil | ⅕ | Glycerin | Glycerin fatty | A | A | A |
| | | | | acid ester | | | |
| Example 3 | Palm oil/rice oil | ⅕ | Glycerin | Glycerin fatty | A | A | A |
| | | | | acid ester | | | |
| Example 4 | Palm oil/safflower oil | ⅕ | Glycerin | Glycerin fatty | B | B | B |
| | | | | acid ester | | | |
| Example 5 | Medium chain fatty acid triglyceride | ⅙ | Glycerin | Glycerin fatty | A | A | A |
| | | | | acid ester | | | |
| Example 6 | Medium chain fatty acid triglyceride | ⅕ | Reduced starch | Glycerin fatty | B | B | B |
| | | | syrup | acid ester | | | |
| Example 7 | Medium chain fatty acid triglyceride | ⅕ | Glycerin | Sucrose fatty | B | B | B |
| | | | | acid ester | | | |
| Example 8 | Medium chain fatty acid triglyceride | ⅕ | Glycerin | Sorbitan fatty | B | B | B |
| | | | | acid ester | | | |
| Example 9 | Medium chain fatty acid triglyceride | ⅕ | Reduced starch | Sucrose fatty | B | B | B |
| | | | syrup | acid ester | | | |
| Example 10 | Palm oil | ⅕ | Reduced starch | Glycerin fatty | B | B | B |
| | | | syrup | acid ester | | | |

In Example 4, the palm oil and safflower oil were used at a mass ratio of 1:1, and the polyunsaturated fatty acid content of the entire oil component was approximately 40% by mass.

The details of the compound shown in Table 2 except the details described above are as follows.

Reduced starch syrup (product name: oligotose H-70, manufactured by Mitsubishi-Chemical Foods Corporation)

Edible fat and oil (safflower oil, product name: Nisshin safflower oil (S) iodine value of 136 to 148, manufactured by The Nisshin Oillio Group, Ltd., polyunsaturated fatty acid content: approximately 70% by mass)

Edible fat and oil (rice oil, product name: rice bran oil, manufactured by TSUNO CO., LTD., polyunsaturated fatty acid content: 33.3% by mass)

Sorbitan fatty acid ester (product name: POEM S-6.5 V: manufactured by Riken Vitamin Co., Ltd.)

Sucrose fatty acid ester (product name: RYOTO Sugar Ester ER-190, manufactured by Mitsubishi-Chemical Foods Corporation)

### Reference Example 1 and Comparative Example 5

Glycerin fatty acid ester (polyglycerin condensed ricinoleic acid ester) and edible fat and oil (oil component) were mixed and dissolved with respect to the amount of antioxidant (ascorbic acid palmitic ester or L-ascorbic acid) shown in Table 3 or Table 4, with the amounts shown in Table 3 or Table 4, and mixed while stirring using a magnetic stirrer, and each oil phase composition was obtained.

The obtained oil phase composition (dispersion) was subjected to a treatment 10 times at a pressure of 20 OMPa by Star Burst Mini (manufactured by Sugino Machine Limited) which is a high-pressure dispersion apparatus, and accordingly, antioxidant dispersions of Reference Example 1 and Comparative Example 5 were obtained.

The evaluation was performed with the obtained antioxidant dispersions of Reference Example 1 and Comparative Example 5 by the evaluation method. The evaluation results are collectively shown in Table 5.

TABLE 3
Oil phase composition (Reference Example 1)

| Component | Used amount (% by mass) |
|---|---|
| Antioxidant | 10 |
| Edible fat and oil | 90 |
| Total (% by mass) | 100 |

TABLE 4
Oil phase composition (Comparative Example 5)

| Component | Used amount (% by mass) |
|---|---|
| Antioxidant | 5 |
| Glycerin fatty acid ester | 10 |
| Edible fat and oil | 85 |
| Total (% by mass) | 100 |

TABLE 5

| | | Content of | | | | | |
|---|---|---|---|---|---|---|---|
| | | antioxidant | | | | | |
| | | with respect to | Oil | | | | |
| | | entire | component | | | CDM test | |
| | | dispersion | (edible fat and | | Emulsion | | Elapse |
| | Antioxidant | (% by mass) | oil) | Emulsifier | stability | Fr | of time |
| Reference | Ascorbic acid | 10 | Palm oil | — | — | — | — |
| Example 1 | palmitic ester | | | | | | |
| Comparative | L-ascorbic acid | 5 | Palm oil | Glycerin | C | A | A |
| Example 5 | | | | fatty acid | | | |
| | | | | ester | | | |

In Reference Example 1, the antioxidant was not sufficient dispersed and an evaluable dispersion was not obtained.

The details of each component used in Reference Example 1 or Comparative Example 5 are shown below.

Edible fat and oil: palm oil (product name, The Nisshin OilliO Group, Ltd., polyunsaturated fatty acid content: 9.2% by mass)

Glycerin fatty acid ester (polyglycerin condensed ricinoleic acid ester, product name: POEM PR300, manufactured by Riken Vitamin Co., Ltd.)

L-ascorbic acid (vitamin C, product name: L-ascorbic acid, manufactured by DSM)

Ascorbic acid palmitic ester (product name: vitamin C palmitate, manufactured by Mitsubishi-Chemical Foods Corporation)

### Examples 11 and 12

Antioxidant dispersions of Examples 11 and 12 were respectively produced in the same manner as in Example 1, except that the following mixed tocopherol as the oil-soluble composition was further added to the list of Example 1, so that the total concentration becomes 0.5% by mass (Example 11) or 1% by mass (Example 12). The emulsified stability evaluation and the CDM test were performed by the same method as described above, and the same effect as in Example 1 was obtained.

Mixed tocopherol (oil-soluble antioxidant, product name: Riken E Oil 800, manufactured by Riken Vitamin Co., Ltd.)

### Example 13

An antioxidant dispersion of Example 13 was produced in the same manner as in Example 1, except that SY-Glyster CR-200 (polyglycerin condensed ricinoleate ester) manufactured by Sakamoto Yakuhin kogyo Co., Ltd. was added to the list of Example 1, instead of polyglycerin condensed ricinoleic acid ester (product name: POEM PR300, manufactured by Riken Vitamin Co., Ltd.). The emulsified stability evaluation and the CDM test were performed by the same method as described above, and the same effect as in Example 1 was obtained.

### Examples 14 and 15

The test was performed in the same manner as in Example 1, except that the content of the emulsifier was changed to 1/20 to 1/10 of that in Example 1. The composition of each phase was shown in Table 6 (Example 14) and Table 7 (Example 15).

TABLE 6

| | |
|---|---|
| Component | Used amount (% by mass) |

Water phase composition

| | |
|---|---|
| Ascorbic acid | 5 |
| Glycerin | 35.5 |
| Water | 18 |

Oil phase composition

| | |
|---|---|
| Glycerin fatty acid ester | 0.5 |
| Edible fat and oil | 41 |
| Total amount of water phase and | 100 |
| oil phase (% by mass) | |

TABLE 7

| | |
|---|---|
| Component | Used amount (% by mass) |

Water phase composition

| | |
|---|---|
| Ascorbic acid | 5 |
| Glycerin | 35 |
| Water | 18 |

Oil phase composition

| | |
|---|---|
| Glycerin fatty acid ester | 1 |
| Edible fat and oil | 41 |
| Total amount of water phase | 100 |
| and oil phase (% by mass) | |

### Example 16

In Example 16, the test was performed in the same manner as in Example 15, except that medium chain fatty acid triglyceride and canola oil (product name: Nisshin canola oil, The Nisshin OilliO Group, Ltd., polyunsaturated fatty acid content: 26.1% by mass (see rapeseed oil in Five-Revised Japanese Food Composition Table)) were used with the amount of 41% by mass at a mass ratio of 1:1, as the edible fat and oil.

### Example 17

In Example 17, the test was performed in the same manner as in Example 15, except that medium chain fatty acid triglyceride and palm oil were used with the amount of 41% by mass at a mass ratio of 1:1, as the edible fat and oil.

### Examples 18 and 19

The test was performed in the same manner as in Example 15, except that the number of kinds of the emulsifier was set as 2, compared to Example 15. In Example 18, the emulsifier added to the oil phase was added, and in Example 19, the emulsifier added to the water phase was added. The composition of each phase is shown in Table 8 (Example 18) and Table 9 (Example 19).

TABLE 8

| | |
|---|---|
| | Used amount |
| Component | (% by mass) |

Water phase composition

| | |
|---|---|
| Ascorbic acid | 5 |
| Glycerin | 30 |
| Water | 18 |

Oil phase composition

| | |
|---|---|
| Glycerin fatty acid ester | 1 |
| Sucrose fatty acid palmitate ester | 5 |
| (HLB value: 1) | |
| Edible fat and oil | 41 |
| Total amount of water phase | 100 |
| and oil phase (% by mass) | |

TABLE 9

| | |
|---|---|
| | Used amount |
| Component | (% by mass) |

Water phase composition

| | |
|---|---|
| Ascorbic acid | 5 |
| Glycerin | 30 |
| Water | 18 |
| Sucrose fatty acid palmitate ester | 5 |
| (HLB value: 16) | |

Oil phase composition

| | |
|---|---|
| Glycerin fatty acid ester | 1 |
| Edible fat and oil | 41 |
| Total amount of water phase and | 100 |
| oil phase (% by mass) | |

The details of the compound other than the details shown in Table 8 and Table 9 are as follows.

Sucrose fatty acid palmitate ester (HLB value: 1) (product name: RYOTO Sugar Ester P-170, manufactured by Mitsubishi-Chemical Foods Corporation)

Sucrose fatty acid palmitate ester (HLB value: 16) (product name: RYOTO Sugar Ester P-1670, manufactured by Mitsubishi-Chemical Foods Corporation)

### Example 20

The test was performed in the same manner as in Example 15, except that ascorbyl palmitate was added and the amount of glycerin was decreased by the increased amount of thereof, compared to Example 15. The composition of each phase is shown in Table 10.

TABLE 10

| | |
|---|---|
| Component | Used amount (% by mass) |

Water phase composition

| | |
|---|---|
| Ascorbic acid | 5 |
| Glycerin | 32 |
| Water | 18 |

Oil phase composition

| | |
|---|---|
| Glycerin fatty acid ester | 1 |
| Edible fat and oil | 41 |
| Ascorbyl palmitate | 3 |
| Total amount of water phase | 100 |
| and oil phase (% by mass) | |

### Example 21

The test was performed in the same manner as in example 20, except that sucrose fatty acid palmitate ester (HLB value: 16) was added and the amount of glycerin was decreased by the increased amount of thereof, compared to Example 20. The composition of each phase is shown in Table 11.

TABLE 11

| | |
|---|---|
| Component | Used amount (% by mass) |

Water phase composition

| | |
|---|---|
| Ascorbic acid | 5 |
| Glycerin | 27 |
| Water | 18 |
| Sucrose fatty acid palmitate ester | 5 |
| (HLB value: 16) | |

Oil phase composition

| | |
|---|---|
| Glycerin fatty acid ester | 1 |
| Edible fat and oil | 41 |
| Ascorbyl palmitate | 3 |
| Total amount of water phase and | 100 |
| oil phase (% by mass) | |

### Example 22

The test was performed in the same manner as in example 20, except that the amount of glycerin was decreased and the amount of edible fat and oil was increased by the decreased amount thereof, compared to Example 20. The composition of each phase is shown in Table 12.

TABLE 12

| | |
|---|---|
| Component | Used amount (% by mass) |

Water phase composition

| | |
|---|---|
| Ascorbic acid | 5 |
| Glycerin | 3 |
| Water | 18 |

Oil phase composition

| | |
|---|---|
| Glycerin fatty acid ester | 1 |
| Edible fat and oil | 70 |
| Ascorbyl palmitate | 3 |
| Total amount of water phase | 100 |
| and oil phase (% by mass) | |

### Example 23

The test was performed in the same manner as in example 15, except that sucrose fatty acid palmitate ester (HLB value: 1) was added and the amount of glycerin was decreased by the increased amount of thereof, compared to Example 15.

TABLE 13
Water phase composition

| Component | Used amount (% by mass) |
|---|---|
| Ascorbic acid | 5 |
| Glycerin | 27 |
| Water | 18 |
| Glycerin fatty acid ester | 1 |
| Edible fat and oil | 41 |
| Sucrose fatty acid palmitate ester | 5 |
| (HLB value: 1) | |
| Ascorbyl palmitate | 3 |
| Total amount of water phase and | 100 |
| oil phase (% by mass) | |

Each of the obtained antioxidant dispersions was evaluated by the method described above. The evaluation results of Examples 14 to 23 are collectively shown in Table 14.

TABLE 14

| | | Mass ratio | Polyhydric | | | | | | | |
|---|---|---|---|---|---|---|---|---|---|---|
| | | of water- | alcohol | | | | | | | |
| | | soluble | compound | | | | | | | |
| | | antioxidant/ | having 5 or | Emulsifier | | | | | CDM test | |
| | Oil component | oil | less carbon | | | HLB value of | Added | Emulsion | | Elapse |
| | (edible fat and oil) | component | atoms | HLB value of 4 or less. | | 11 or more | antioxidant | stability | Fr | of time |
| Example 14 | Medium chain fatty acid | 5/41 | Glycerin | Glycerin | — | — | — | A | A | A |
| | triglyceride | | | fatty acid | | | | | | |
| | | | | ester | | | | | | |
| Example 15 | Medium chain fatty acid | 5/41 | Glycerin | Glycerin | — | — | — | A | A | A |
| | triglyceride | | | fatty acid | | | | | | |
| | | | | ester | | | | | | |
| Example 16 | Medium chain fatty acid | 5/41 | Glycerin | Glycerin | — | — | — | A | A | A |
| | triglyceride/canola oil | | | fatty acid | | | | | | |
| | | | | ester | | | | | | |
| Example 17 | Medium chain fatty acid | 5/41 | Glycerin | Glycerin | — | — | — | A | A | A |
| | triglyceride/palm oil | | | fatty acid | | | | | | |
| | | | | ester | | | | | | |
| Example 18 | Medium chain fatty acid | 5/41 | Glycerin | Glycerin | Sucrose fatty | — | — | A | A | A |
| | triglyceride | | | fatty acid | acid | | | | | |
| | | | | ester | palmitate | | | | | |
| | | | | | ester | | | | | |
| Example 19 | Medium chain fatty acid | 5/41 | Glycerin | Glycerin | — | Sucrose fatty | — | A | A | A |
| | triglyceride | | | fatty acid | | acid | | | | |
| | | | | ester | | palmitate | | | | |
| | | | | | | ester | | | | |
| Example 20 | Medium chain fatty acid | 5/41 | Glycerin | Glycerin | — | — | Ascorbyl | A | A | A |
| | triglyceride | | | fatty acid | | | palmitate | | | |
| | | | | ester | | | | | | |
| Example 21 | Medium chain fatty acid | 5/41 | Glycerin | Glycerin | — | Sucrose fatty | Ascorbyl | A | A | A |
| | triglyceride | | | fatty acid | | acid | palmitate | | | |
| | | | | ester | | palmitate | | | | |
| | | | | | | ester | | | | |
| Example 22 | Medium chain fatty acid | 1/14 | Glycerin | Glycerin | — | — | Ascorbyl | A | A | A |
| | triglyceride | | | fatty acid | | | palmitate | | | |
| | | | | ester | | | | | | |
| Example 23 | Medium chain fatty acid | 5/41 | Glycerin | Glycerin | Sucrose fatty | — | Ascorbyl | A | A | A |
| | triglyceride | | | fatty acid | acid | | palmitate | | | |
| | | | | ester | palmitate | | | | | |
| | | | | | ester | | | | | |

The contents of JP2017-078150A filed on Apr. 11, 2017 and contents of JP2017-160072A filed on Aug. 23, 2017 are incorporated herein by reference.

All of the documents, the patent applications, and the technology standards described in the specification are incorporated here by reference.

What is claimed is:

1. An antioxidant dispersion comprising:
an oil component having a polyunsaturated fatty acid content of less than 50% by mass;
a water-soluble antioxidant;
at least one kind of compound selected from the group consisting of a polyhydric alcohol compound having 5 or less carbon atoms and reduced starch syrup;
an emulsifier; and
water.

2. The antioxidant dispersion according to claim 1,
wherein the emulsifier contains a compound having an HLB value of 4.0 or less.

3. The antioxidant dispersion according to claim 2,
wherein the emulsifier is a mixture of two kinds of compound having an HLB value of 4.0 or less or a mixture of a compound having an HLB value of 4.0 or less and a compound having an HLB value of 11.0 or more.

4. The antioxidant dispersion according to claim 1,
wherein a content of the emulsifier is 0.5% by mass to 20% by mass with respect to a total mass of the antioxidant dispersion.

5. The antioxidant dispersion according to claim 1,
wherein a content of the emulsifier is 3% by mass to 20% by mass with respect to a total mass of the antioxidant dispersion.

6. The antioxidant dispersion according to claim 1,
wherein the oil component is at least one kind of compound selected from the group consisting of palm oil, rice oil, rapeseed oil, olive oil, sesame oil, safflower oil, coconut oil, and medium chain fatty acid triglyceride.

7. The antioxidant dispersion according to claim 1,
wherein the oil component is at least one kind of compound selected from the group consisting of palm oil, rice oil, rapeseed oil, and medium chain fatty acid triglyceride.

8. The antioxidant dispersion according to claim 1,
wherein the polyhydric alcohol compound having 5 or less carbon atoms is at least one kind of compound selected from the group consisting of glycerin, erythritol, threitol, arabinitol, xylitol, and libitol.

9. The antioxidant dispersion according to claim 1,
wherein the emulsifier is glycerin fatty acid ester.

10. The antioxidant dispersion according to claim 1,
wherein a content of the water-soluble antioxidant is 30% by mass or less with respect to a total mass of the oil component.

11. The antioxidant dispersion according to claim 1, further comprising:
an oil-soluble antioxidant.

12. The antioxidant dispersion according to claim 11,
wherein the oil-soluble antioxidant contains ascorbyl palmitate or tocopherol.
